(19) United States
(12) Patent Application Publication
(10) Pub. No.: US 2022/0370470 A1
(43) Pub. Date: Nov. 24, 2022

(54) METHYLTHIONINIUM FOR USE IN THE TREATMENT OF SYNAPTOPATHIES

(71) Applicant: WisTa Laboratories Ltd., Singapore (SG)

(72) Inventors: Gernot Riedel, Aberdeen, Aberdeenshire (GB); Charles Robert Harrington, Aberdeen, Aberdeenshire (GB); Claude Michel Wischik, Aberdeen, Aberdeenshire (GB)

(73) Assignee: WisTa Laboratories Ltd., Singapore (SG)

(21) Appl. No.: 17/620,966

(22) PCT Filed: Jun. 29, 2020
(86) PCT No.: PCT/EP2020/068306

§ 371 Date: Dec. 20, 2021

(30) Foreign Application Priority Data
Jul. 2, 2019 (GB) 1909506.6

(51) Int. Cl. A61K 31/5415; A61K 31/445; A61K 31/27; A61K 31/55; A61K 31/13; A61P 25/18; A61P 25/24; A61P 25/16; A61P 27/06; A61P 25/08; A61P 25/28

(52) U.S. Cl. CPC A61K 31/5415; A61K 31/445; A61K 31/27; A61K 31/55; A61K 31/13; A61P 25/18; A61P 25/24; A61P 25/16; A61P 27/06; A61P 25/08; A61P 25/28

(57) ABSTRACT

The present invention relates generally to methods and materials for treating synaptopathies, based on the use of Leu-co-methylthioninium acid salts, which are disclosed herein to increase synaptophysin levels in various brain regions at therapeutically relevant doses both in animal models of neurodegenerative disease, and in normal animals.

### TECHNICAL FIELD

The present invention relates generally to methods and materials for treating synaptopathies.

### BACKGROUND ART

Synapses are integral components of neurons and allow an organized flux of information in the brain. The emergence, diversification, and specialization of synapses played a central role in the evolution of higher brain functions and cognition in vertebrates. On the one hand, modulation of synapse activity constitutes a major strategy to control brain homeostasis. On the other hand, slight but persistent perturbations in synapse physiology can result in major defects that may manifest as brain disorders.

Synaptic vesicle (SV)-mediated transmitter release is the main mechanism of neuronal information transfer. SVs are characterized by a very specific polypeptide composition to facilitate this tightly-regulated process.

Synaptophysin is an abundant integral membrane glycoprotein of SVs, with four transmembrane domains and a unique cytoplasmic tail rich in proline, glycine, and tyrosine.

Synaptophysin has been implicated in the regulation of neurotransmitter release and synaptic plasticity and in the biogenesis and recycling of SV. Increases in synaptophysin expression have been found to correlate with long-term potentiation, suggesting that the regulation of synaptophysin expression may contribute to the mechanisms underlying learning and memory.

Aberrant synaptophysin expression has been associated with neurodegenerative diseases and psychiatric disorders. Elimination of synaptophysin in mice is reported to create behavioral changes such as increased exploratory behavior, impaired object novelty recognition, and reduced spatial learning (Schmitt, U., et al. “Detection of behavioural alterations and learning deficits in mice lacking synaptophysin.” Neuroscience 162.2 (2009): 234-243).

The term ‘synaptopathy’ has been used to refer to brain disorders that have arisen from synaptic dysfunction. There is now evidence for the importance of synapse dysfunction as a major determinant of several neurodevelopmental diseases (e.g. schizophrenia, major depression, autism spectrum disorders (ASD), Down syndrome, startle disease, and epilepsy), neurological diseases (e.g. dystonia, levodopa-induced dyskinesia, and ischemia) and neurodegenerative diseases (e.g. Alzheimer and Parkinson disease) (Lepeta et al., 2016).

US20020040032 relates to a method of increasing the synthesis and/or secretion of synaptophysin which comprises administering to a patient with a neurological disease or a patient at risk of developing a neurological disease an effective quantity of a purine derivative or analogue, a tetrahydroindolone derivative or analogue, or a pyrimidine derivative or analogue. Examples of neurological diseases referred to include neurodegenerative disease such as Alzheimer's disease or a neurodevelopmental disorder such as Down's syndrome.

Nevertheless it can be seen that the characterisation of further compounds which can modulate, and in particular increase, synaptophysin levels in the brain would provide a contribution to the art.

### DISCLOSURE OF THE INVENTION

The present inventors have unexpectedly found that Leuco-methylthioninium acid salts (referred to herein as “LMTX” salts) can increase synaptophysin levels in various brain regions at therapeutically relevant doses both in animal models of neurodegenerative disease, and in normal (wild-type) animals.

The present findings imply new utilities for LMTX salts at therapeutically relevant doses for use in the treatment of synaptopathies.

Bis(hydromethanesulfonate) (LMTM; USAN name hydromethylthionine mesylate) is being developed as a treatment targeting pathological aggregation of tau protein in AD (Wischik et al., 2018). The methylthioninium (MT) moiety can exist in oxidised (MT⁺) and reduced (LMT) forms. LMTM is a stabilised salt of LMT which has much better pharmaceutical properties than the oxidised MT⁺ form (Baddeley et al., 2015; Harrington et al., 2015). We have reported recently that LMT rather than MT⁺ is the active species blocking tau aggregation in vitro (Al-Hilaly et al., 2018). LMT blocks tau aggregation in vitro in cell-free and cell-based assays (Harrington et al., 2015; Al-Hilaly et al., 2018), and reduces tau aggregation pathology and associated behavioural deficits in tau transgenic mouse models in vivo at clinically relevant doses (Melis et al., 2015a). LMT also disaggregates the tau protein of the paired helical filaments (PHFs) isolated from AD brain tissues converting the tau into a form which becomes susceptible to proteases (Wischik et al., 1996; Harrington et al., 2015).

Although LMTM given orally produces brain levels sufficient for activity in vitro and in vivo (Baddeley et al., 2015), it had minimal apparent efficacy if taken as an add-on to symptomatic treatments in two large Phase 3 AD clinical trials (Gauthier et al., 2016; Wilcock et al., 2018). In subjects receiving LMTM as monotherapy, however, treatment produced marked slowing of cognitive and functional decline, reduction in rate of progression of brain atrophy measured by MRI and reduction in loss of glucose uptake measured by FDG-PET (Gauthier et al., 2016; Wilcock et al., 2018). When these outcomes were analysed in combination with population pharmacokinetic data available from subjects participating in the trials, LMTM was found to produce concentration-dependent effects whether taken alone or in combination with symptomatic treatments such as acetylcholinesterase inhibitors. However, the treatment effects in monotherapy subjects were substantially larger than in those taking LMTM in combination with symptomatic treatments.

LMTM and other Leuco-methylthioninium bis-protic acid salts have been suggested for the treatment of various diseases, impairments and pathologies in several publications e.g. WO2007/110627, WO2008/155533, WO2009/044127, WO2012/107706, WO2018019823 and WO2018041739.

The present studies were undertaken with the aim of understanding the mechanisms responsible for the reduced efficacy of LMTM as an add-on to symptomatic treatments discussed above. In these studies a well-characterised tau transgenic mouse model (Line 1, “L1”; (Melis et al., 2015b)) was compared with wild-type mice.

One conclusion from the present studies is that homeostatic mechanisms downregulate multiple neuronal systems at different levels of brain function to compensate for the chronic pharmacological activation induced by prior symptomatic treatments. Compared with LMTM given alone, the effect of this downregulation is to reduce neurotransmitter release, levels of synaptic proteins, mitochondrial function and behavioural benefits if LMTM is given against a background of chronic prior exposure to acetylcholinesterase inhibitor.

Unexpectedly, however, the studies also revealed that LMTX salts increased synaptophysin levels in various brain regions at therapeutically relevant doses both in the L1 and wild-type mice. This finding offers new utilities for LMTX in diseases of synaptic dysfunction.

Thus in one aspect there is provided a method of increasing the level of synaptophysin in the brain of a mammalian subject, the method comprising orally administering MT to the subject per day,
wherein the MT compound is an LMTX compound of the following formula:

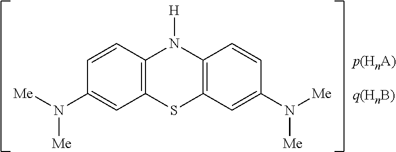

wherein each of HₙA and HₙB (where present) are protic acids which may be the same or different,
and wherein p=1 or 2; q=0 or 1; n=1 or 2; (p+q)×n=2.

The subject may be selected to be one who is in need of an increased level of synaptophysin.

The subject may be a human subject or patient having, or being at risk of developing, a synaptopathy.

The subject may be a human subject or patient having, or being at risk of developing, a neurodevelopmental, neurological, or neurodegenerative disease.

The increase levels may be in multiple brain regions. For example, temporal lobes, important for memory, are affected commonly in epilepsy. Schizophrenia is often considered as a neurodevelopmental disorder; by imaging it is characterised by generalised cortical loss and ventricular enlargement with smaller thalamus and temporal lobes and enlarged caudate nucleus. However, due to brain connectivity, the effect of synaptic dysfunction may be exerted in multiple brain regions.

The findings of the present inventors have implication for the novel uses of LMTX compounds in neurodevelopmental, neurological and neurodegenerative diseases in which it has not previously been indicated. They further have implications for use in patient sub-groups in diseases where LMTX has previously been suggested for use, which sub-groups are those where synaptic dysfunction is more specifically implicated.

Thus another aspect of the invention provides methods of therapeutic treatment of a disorder in a subject. Appropriate disorders are listed as follows. In particular, “synaptopathies” in which LTMX may have utility include:
Schizophrenia
Depression
Epilepsy
Startle syndrome (Tourette's syndrome and anxiety disorders)
Autism spectrum disorders (ASD) (autism, Asperger syndrome, pervasive developmental disorder not otherwise specified (PDD-NOS), and childhood disintegrative disorder)
Focal hand dystonia
Cerebral ischemia
Experimental allergic encephalitis (EAE)
Multiple sclerosis (MS)
Glaucoma

There is much evidence on the role of synaptophysin in AD. Synapses are considered the earliest site of pathology, and synaptic loss is the best pathological correlate of cognitive impairment in subjects with AD (Terry et al., 1991). Synaptic abnormalities in the hippocampus correlate with the severity of neuropathology and memory deficit in individuals with AD, and this defect may predate neuropsychological evidence for cognitive impairment early in AD (Sze et al., 1997).

Furthermore genome-Wide Association Studies (GWAS) have identified >20 loci associated with late-onset AD, which were grouped in three major biological pathways—lipid metabolism, immune system, and synaptic dysfunction/cell membrane processes (Van Giau et al., 2019; Verheijen and Sleegers, 2018, Understanding Alzheimer Disease at the Interface between Genetics and Transcriptomics. Trends Genet. 34:434-447).

Synaptic density can be detected in vivo in AD using positron emission tomography imaging (Chen et al., 2018, Assessing synaptic density in Alzheimer disease with synaptic vesicle glycoprotein 2a positron emission tomographic imaging. JAMA Neurol. 75:1215-1224). This may be used both for patient selection criteria and as an outcome measure for trials of disease-modifying therapies, particularly those targeted at the preservation and restoration of synapses. For example patients may be selected demonstrating a reduction in hippocampal SV2A specific binding of at least 30% compared with cognitively normal participants, as assessed by ¹¹C-UCB-J-PET BPND (see Chen, 2018).

Thus subjects in sub-groups having late-onset AD, particularly those characterised as having synaptic dysfunction, form a further target patient group of the present invention.

Lysosomal storage diseases (LSDs) are a group of about 70 rare inherited metabolic disorders that result from defects in lysosomal function (e.g. Parenti, Andria and Ballabio, 2015, Lysosomal Storage Diseases: From Pathophysiology to Therapy. Ann. Rev. Med. 66:471-486; Lloyd-Evans and Haslett, 2016, The lysosomal storage disease continuum with ageing-related neurodegenerative disease. Ageing Research Reviews 32:104-121). Lysosomes digest large molecules within cells and pass the fragments on to other parts of the cell for recycling. Where enzymes in this process are defective, large molecules accumulate within the cell leading to cellular death. No cures for lysosomal storage diseases are known, and treatment is mostly symptomatic.

The LSDs are generally classified by the nature of the primary stored material involved, and can be broadly broken into the following disorders: Lipid storage disorders; Sphingolipidoses, including Gaucher's and Niemann-Pick diseases; Gangliosidosis (including Tay-Sachs disease); Leukodystrophies; Mucopolysaccharidoses (including Hunter syndrome and Hurler disease); Glycoprotein storage disorders; Mucolipidoses; Glycogen storage disease type II (Pompe disease); and Cystinosis.

Alternatively, LSDs may be classified according to the protein targets, e.g.: defects in various lysosomal enzymes (including Tay-Sachs disease, I-cell disease, and Sphingolipidoses, e.g., Krabbe disease, gangliosidosis, Gaucher, Niemann Pick disease, metachromatic leukodystrophy); posttranslational modification of sulphatases (multiple sulphatase deficiency); enzyme protecting proteins (e.g. defective cathepsin A in galactosialidosis); transmembrane proteins (e.g. sphingolipid activator proteins and Sialin in Salla disease) (see e.g. http://www.lysosomaldiseasenetwork.org/official-list-lysosomal-diseases).

Lysosomal storage disorders (LSDs) often show a neurodegenerative course and there is no cure to treat the central nervous system in LSDs. Moreover, the mechanisms driving neuronal degeneration in these pathological conditions remain largely unknown. In mouse models of LSDs, impaired lysosomal activity causes perikaryal accumulation of insoluble α-synuclein and increased proteasomal degradation of cysteine string protein α (CSPα) (Sambri et al., 2017, Lysosomal dysfunction disrupts presynaptic maintenance and restoration of presynaptic function prevents neurodegeneration in lysosomal storage diseases. EMBO Molecular Medicine 9:112-132). As a result, the availability of both α-synuclein and CSPα at nerve terminals strongly decreases, thus inhibiting SNARE complex assembly and synaptic vesicle recycling.

Neurodegeneration in LSDs may be slowed down by re-establishing presynaptic functions. Thus improved synapse maintenance in accordance with the disclosure herein provides one means for treating or mitigating the effects of LSDs.

WO2012/107706 and WO2018/0198823 both discuss the utility of LMTX compounds, in their capacity as tau aggregation inhibitors, in treating lysosomal storage disorders associated with tau pathology. Both Niemann-Pick Type C disease (NPC) and Sanfilippo syndrome type B are referred to (see also Suzuki et al. 1995, Neurofibrillary tangles in Niemann-Pick type C, Acta Neuropathol., 89(3) 227-238; Ohmi et al. 2009 Sanfilippo syndrome type B, a lysosomal storage disease, is also a tauopathy. Proceedings of the National Academy of Sciences 106:8332-8337).

However in the light of the present disclosure it can be seen that other types of LSD, even those not associated with tau pathology, may be improved by the use of LMTX type compounds. Thus treatment of an LSD, optionally not a tauopathy, for example not NPC or Sanfilippo syndrome type B, forms one aspect of the invention. Examples include:

Gaucher's disease; Tay-Sach; Leukodystrophies; Mucopolysaccharidoses (including Hunter syndrome and Hurler disease); Glycoprotein storage disorders; Mucolipidoses; Glycogen storage disease type II (Pompe disease); Cystinosis; I-cell disease; Krabbe disease; gangliosidosis; metachromatic leukodystrophy; multiple sulphatase deficiency; galactosialidosis; Salla disease.

Other examples include:

Activator deficiency, GM2-gangliosidosis; GM2-gangliosidosis, AB variant; alpha-mannosidosis; beta-mannosidosis; aspartylglucosaminuria; lysosomal acid lipase deficiency; Chanarin-Dorfman syndrome; Danon disease; Fabry disease; Farber disease; Farber lipogranulomatosis; fucosidosis; galactosialidosis (combined neuraminidase & beta-galactosidase deficiency); GM1-gangliosidosis; Mucopolysaccharidoses disorders; MPS I, Hurler syndrome; MPS I, Hurler-Scheie syndrome; MPS I, Scheie syndrome; MPS II, Hunter syndrome; MPS II, Hunter syndrome; Morquio syndrome, type A/MPS IVA; Morquio syndrome, type B/MPS IVB; MPS IX hyaluronidase deficiency; MPS VI Maroteaux-Lamy syndrome; MPS VII Sly syndrome; mucolipidosis I, sialidosis; Pseudo-Hurler polydystrophy/mucolipidosis type III; mucolipidosis IIIC/ML III GAMMA; mucolipidosis type IV; Neuronal Ceroid Lipofuscinoses; CLN6 disease—Atypical Late Infantile, Late-Onset variant, Early Juvenile; Batten-Spielmeyer-Vogt/Juvenile NCL/CLN3 disease; Finnish Variant Late Infantile CLN5; Jansky-Bielschowsky disease/Late infantile CLN2/TPP1 Disease; Kufs/Adult-onset NCL/CLN4 disease; Northern Epilepsy/variant late infantile CLN8; Santavuori-Haltia/Infantile CLN1/PPT disease; Pycnodysostosis; Sandhoff disease/GM2 gangliosidosis; Sandhoff disease/GM2 gangliosidosis; Sandhoff disease/GM2 Gangliosidosis; Schindler disease; Kanzaki disease; infantile free sialic acid storage disease (ISSD); spinal muscular atrophy with progressive myoclonic epilepsy (SMAPME); Christianson syndrome; Lowe oculocerebrorenal syndrome; Charcot-Marie-Tooth type 4J, CMT4J; Yunis-Varon syndrome; bilateral temporooccipital polymicrogyria (BTOP); X-linked hypercalciuric nephrolithiasis, Dent-1; Dent disease 2.

Another aspect of the present invention pertains to a methylthioninium (MT) containing LTMX compound as described herein for use the methods as described above e.g. of methods of increasing the level of synaptophysin in the brain of a mammalian subject, or methods of treating the specified diseases described herein.

Another aspect of the present invention pertains to use of a methylthioninium (MT) containing LTMX compound as described herein in the manufacture of a medicament for use in the methods above e.g. methods of increasing the level of synaptophysin in the brain of a mammalian subject, or methods of treating the specified diseases described herein.

With particular (but non-limiting) relevance to cognitive disorders, the subjects may be those who are not receiving, and have not previously received, treatment with acetylcholinesterase inhibitors (AChEIs) or the N-methyl-D-aspartate receptor antagonist memantine. Examples of acetylcholinesterase inhibitors include Donepezil (Aricept™), Rivastigmine (Exelon™) or Galantamine (Reminyl™). An example of an NMDA receptor antagonist is Memantine (Ebixa™, Namenda™).

For example the subject group may be entirely naïve to these other treatments, and have not historically received one or both of them.

However the subject group may have historically received one or both of these treatments, but ceased that medication at least 1, 2, 3, 4, 5, 6, 7 days, or 2, 3, 4, 5, 6, 7, 8, 12, or 16 weeks, or more preferably at least 1, 2, 3, 4, 5 or 6 months etc. prior to treatment with an MT compound according to the present invention.

Any aspect of the present invention may include the active step of selecting the subject group according to these criteria.

The term “treatment” includes “combination” therapeutic treatments, in which two or more treatments to treat the relevant disease are are combined, for example, sequentially or simultaneously.

In combination treatments, the agents (i.e., an MT compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

An example of a combination treatment of the invention would be use of the MT compound with a treatment for the same disease previously known in the art.
Schizophrenia: therapeutics for treatment of schizophrenia are typically anti-psychotics that generally affect dopamine or serotonin neurotransmission. First generation anti-psychotics include chlorpromazine, fluphenazine, haloperidol and perphenazine. Second generation anti-psychotics have less side-effects and include clozapine, olanzapine, quetiapine and risperidone.
Depression may involve treatment with second generation anti-psychotics.
Epilepsy may be treated by various anti-epileptic drugs, whose action is aimed at reducing excessive electrical activity in the brain. These include sodium valproate (Epilin), levitiracetam, phenobarbital, topiramate and zonisamide.
Startle syndrome (Tourette's syndrome and anxiety disorders). The classes of medication with the most proven efficacy in treating tics are typical and atypical neuroleptics including risperidone (Risperdal), ziprasidone (Geodon), haloperidol (Haldol), pimozide (Orap) and fluphenazine (Prolixin).
Autism spectrum disorders (ASD). More than half of U.S. children diagnosed with ASD are prescribed psychoactive drugs or anticonvulsants, with the most common drug classes being antidepressants, stimulants, and antipsychotics. Only the antipsychotics have clearly demonstrated efficacy. Selective serotonin reuptake inhibitors (SSRIs) and dopamine blockers can reduce some maladaptive behaviors associated with ASD.
Focal hand dystonia: This condition is often treated with injections of botulinum neurotoxin A which reduces the symptoms of the disorder but is not a cure. Anticholinergics such as Artane may be prescribed for off-label use.
Cerebral ischemia. Alteplase is a thrombolytic drug. It is a tissue plasminogen activator approved by the US Food and Drug Administration for the treatment of acute ischemic stroke.
Experimental allergic encephalitis (EAE) is an autoimmune demyelinating condition which may be treated by therapies used to treat multiple sclerosis.
Multiple sclerosis (MS) is a chronic inflammatory demyelinating condition that may be treated with dimethyl fumarate, fingolimod (a sphingosine-1-phosphate receptor modulator), natilizumab (Tysabri), alemtuzumab, ocrelizumab, interferons and glatirimer acetate.
Glaucoma: Several classes of medications may be used to treat glaucoma. Prostaglandin analogs, such as latanoprost, bimatoprost and travoprost, increase uveoscleral outflow of aqueous humor. Topical beta-adrenergic receptor antagonists, such as timolol, levobunolol, and betaxolol, decrease aqueous humor production by the epithelium of the ciliary body. Alpha2-adrenergic agonists, such as brimonidine and apraclonidine, work by a dual mechanism, decreasing aqueous humor production and increasing uveoscleral outflow. Less-selective alpha agonists, such as epinephrine, decrease aqueous humor production through vasoconstriction of ciliary body blood vessels. Miotic agents (parasympathomimetics), such as pilocarpine, work by contraction of the ciliary muscle, opening the trabecular meshwork and allowing increased outflow of the aqueous humour. Echothiophate, an acetylcholinesterase inhibitor, is used in chronic glaucoma. Carbonic anhydrase inhibitors, such as dorzolamide, brinzolamide, and acetazolamide, lower secretion of aqueous humor by inhibiting carbonic anhydrase in the ciliary body.
LSDs: Treatments include enzyme replacement therapy, small molecule pharmacological chaperones, or gene therapy strategies for correcting genetic mutation (Bruni S, Loschi L, Incerti C, Gabrielli O, Coppa G V. Update on treatment of lysosomal storage diseases. Acta Myol. 2007; 26(1):87-92.); Parenti, Giancarlo, et al. “New strategies for the treatment of lysosomal storage diseases.” International journal of molecular medicine 31.1 (2013): 11-20.

The use of the MT compound in the methods or uses described herein in combination with any of these other therapeutics forms an aspect of the present invention.

In other embodiments the treatment is a “monotherapy”, which is to say that the MT-containing compound is not used in combination (within the meaning discussed above) with another active agent.

As noted above, it is specifically envisaged that administration of the MT-compound may be commenced in subjects who have not previously received (and are not currently receiving) with AChEIs or memantine.

However such AChEIs or memantine treatment may optionally be started or re-started after commencement of treatment with the MT compound, for example after around 3 months of treatment with the MT compound. That may be desirable, for example, in relation to subjects being treated for late-onset AD (synaptic dysfunction).

### LMTX Compounds

Preferably the MT compound is an “LMTX” compound of the type described in WO2007/110627 or WO2012/107706.

Thus the compound may be selected from compounds of the following formula, or hydrates or solvates thereof:

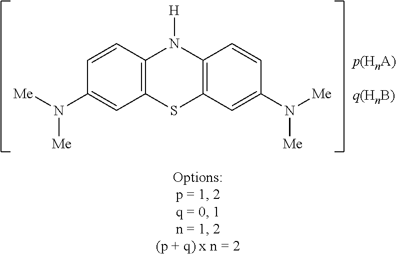

Each of HₙA and HₙB (where present) are protic acids which may be the same or different.

By “protic acid” is meant a proton (H⁺) donor in aqueous solution. Within the protic acid A⁻ or B⁻ is therefore a conjugate base. Protic acids therefore have a pH of less than 7 in water (that is the concentration of hydronium ions is greater than 10⁻⁷ moles per litre).

In one embodiment the salt is a mixed salt that has the following formula, where HA and HB are different mono-protic acids:

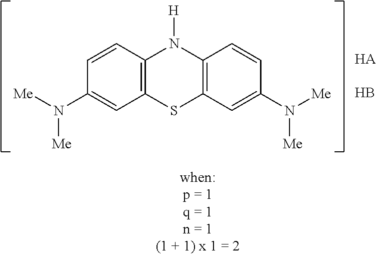

However preferably the salt is not a mixed salt, and has the following formula:

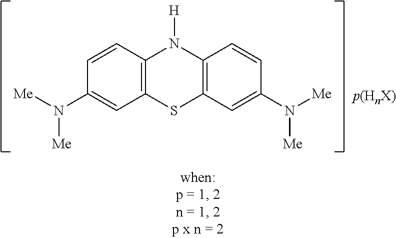

wherein each of HₙX is a protic acid, such as a di-protic acid or mono-protic acid.

In one embodiment the salt has the following formula, where H₂A is a di-protic acid:

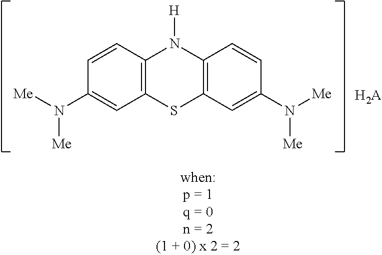

Preferably the salt has the following formula which is a bis monoprotic acid:

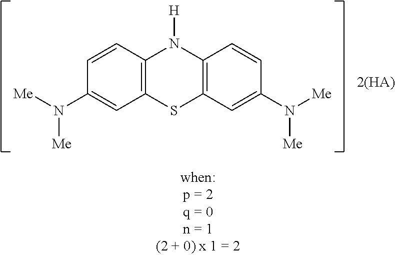

Examples of protic acids which may be present in the LMTX compounds used herein include:

Inorganic acids: hydrohalide acids (e.g., HCl, HBr), nitric acid (HNO₃), sulphuric acid (H₂SO₄)

Organic acids: carbonic acid (H₂CO₃), acetic acid (CH₃COOH), methanesulfonic acid, 1,2-ethanedisulfonic acid, ethansulfonic acid, naphthalenedisulfonic acid, p-toluenesulfonic acid,

Preferred acids are monoprotic acid, and the salt is a bis(monoprotic acid) salt.

A preferred MT compound is LMTM:

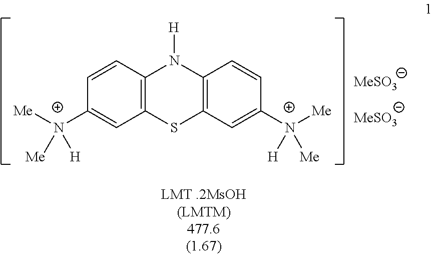

The anhydrous salt has a molecular weight of around 477.6. Based on a molecular weight of 285.1 for the LMT core, the weight factor for using this MT compound in the invention is 1.67. By “weight factor” is meant the relative weight of the pure MT containing compound vs. the weight of MT which it contains.

Other weight factors can be calculated for example MT compounds herein, and the corresponding dosage ranges can be calculated therefrom.

Therefore the invention embraces a total daily dose of around 2-100 mg/day of LMTM.

More preferably around 6 to 12 mg/day of LMTM total dose is utilised, which corresponds to about 3.5 to 7 mg MT.

Other example LMTX compounds are as follows. Their molecular weight (anhydrous) and weight factor is also shown:

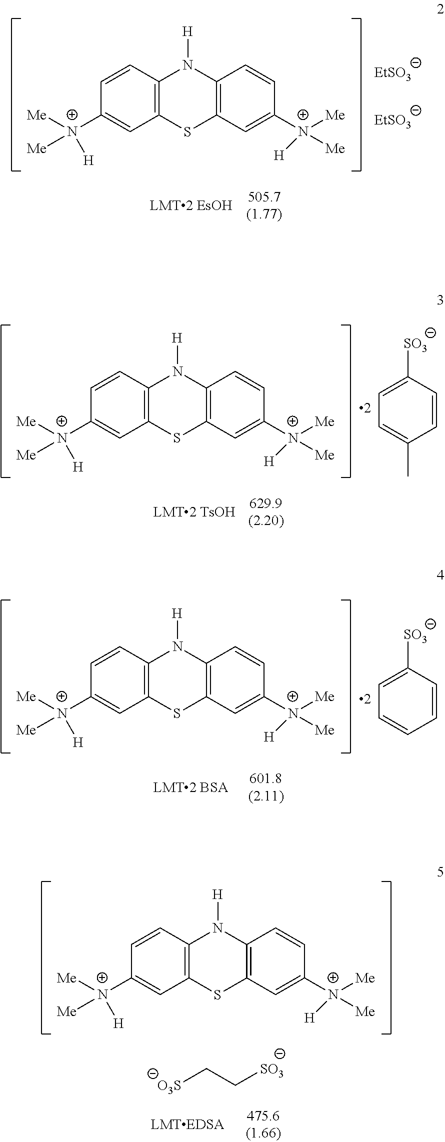

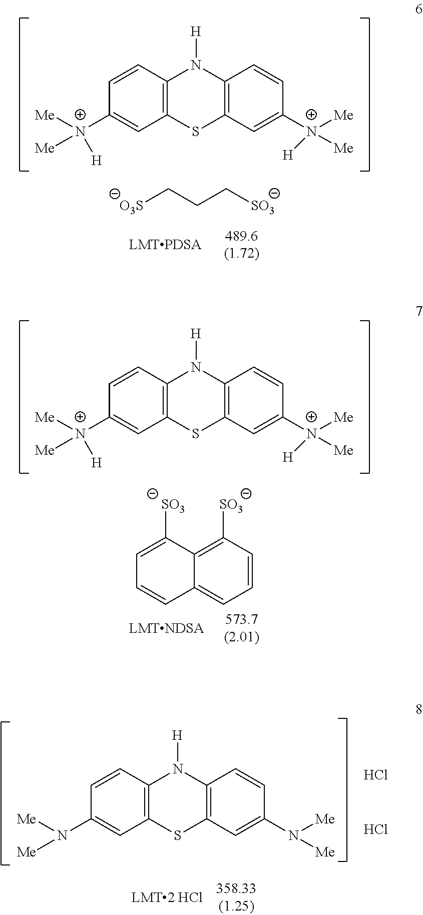

In the various aspects of the invention described herein (as they relate to an MT-containing compound) this may optionally be any of those compounds described above:

In one embodiment, it is compound 1.

In one embodiment, it is compound 2.

In one embodiment, it is compound 3.

In one embodiment, it is compound 4.

In one embodiment, it is compound 5.

In one embodiment, it is compound 6.

In one embodiment, it is compound 7.

In one embodiment, it is compound 8.

Or the compounds may be a hydrate, solvate, or mixed salt of any of these.

Based on the results herein, and prior and concurrent results using LMTM in the treatment of disease, it can be concluded that MT dosages in the range 2-80 or 100 mg/day could be beneficial for the synaptopathy diseases described herein.

More specifically further analysis of the concentration-response for LMTM in relation to the treatment of disease supports the proposition that a preferred dose is at least 2 mg/day, and doses in the range 20-40 mg/day, or 20-60 mg/day would be expected to maximise the cognitive benefit while nevertheless maintaining a desirable profile in relation to being well tolerated with minimal side-effects.

Thus in one embodiment the total MT dose may be from around any of 2, 2.5, 3, 3.5, 4 mg to around any of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 mg.

An example dosage is 2 to 60 mg e.g. 20, 30, 40, 50, or 60 mg.

An example dosage is 20 to 40 mg.

Further example dosages are 8 or 16 or 24 mg/day.

The subject of the present invention may be an adult human, and the dosages described herein are premised on that basis (typical weight 50 to 70 kg). If desired, corresponding dosages may be utilised for subjects outside of this range by using a subject weight factor whereby the subject weight is divided by 60 kg to provide the multiplicative factor for that individual subject.

As will be appreciated by those skilled in the art, for a given daily dosage, more frequent dosing will lead to greater accumulation of a drug.

The present inventors have derived estimated accumulation factors for MT as follows:

| | Observed plasma | Relative |
|---|---|---|
| Dosing | accumulation for MT | accumulation |
| Once daily | 1.29^{extrapolated} | 1 |
| Twice daily | 1.47 | 1.13 |
| Three-times daily | 1.65 | 1.28 |

For example, considering a total daily dose of 3.5 to 7 mg MT:

When given as a single daily dose, this may equate to an accumulation of MT in plasma of 4.5 to 8

When split b.i.d., this may equate to an accumulation of MT in plasma of 5.1 to 10.3

When split t.i.d., this may equate to an accumulation of MT in plasma of 5.8 to 11.6

Therefore in certain embodiments of the invention, the total daily dosed amount of MT compound may be lower, when dosing more frequently (e.g. twice a day [b.i.d.] or three times a day [t.i.d.]).

In one embodiment, LMTM is administered around 9 mg/once per day; 4 mg b.i.d.; 2.3 mg t.i.d (based on weight of LMTM).

In one embodiment, LMTM is administered around 34 mg/once per day; 15 mg b.i.d.; 8.7 mg t.i.d (based on weight of LMTM).

The MT compound of the invention, or composition comprising it, is administered to a subject orally.

In some embodiments, the MT compound is administered as a composition comprising the LMTX compound as described herein, and a pharmaceutically acceptable carrier, diluent, or excipient.

The term “pharmaceutically acceptable,” as used herein, pertains to compounds, ingredients, materials, compositions, dosage forms, etc., which are suitable for use in contact with the tissues of the subject in question without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Compositions comprising LMTX salts are described in several publications e.g. WO2007/110627, WO2009/044127, WO2012/107706, WO2018019823 and WO2018041739.

In some embodiments, the composition is a composition comprising at least one LMTX compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994.

In some embodiments, the composition is a tablet.

In some embodiments, the composition is a capsule.

In some embodiments, said capsules are gelatine capsules.

In some embodiments, said capsules are HPMC (hydroxypropylmethylcellulose) capsules.

In some embodiments, the amount of MT in the unit 2 to 60 mg.

In some embodiments, the amount of MT in the unit 10 to 40, or 10 to 60 mg.

In some embodiments, the amount of MT in the unit 20 to 40, or 20 to 60 mg.

An example dosage unit may contain 2 to 10 mg of MT.

A further example dosage unit may contain 2 to 9 mg of MT.

A further example dosage unit may contain 3 to 8 mg of MT.

A further preferred dosage unit may contain 3.5 to 7 mg of MT.

A further preferred dosage unit may contain 4 to 6 mg of MT.

In some embodiments, the amount is about 2, 2.5, 3, 3.5, 4, 5, 6, 7, 8, 9, 10, 11, 12, 13, 14, 15, 16, 17, 18, 19 or 20 mg of MT.

Using the weight factors described or explained herein, one skilled in the art can select appropriate amounts of an MT containing compound to use in oral formulations.

As explained above, the MT weight factor for LMTM is 1.67. Since it is convenient to use unitary or simple fractional amounts of active ingredients, non-limiting example LMTM dosage units may include about 3, 3.5, 4, 5, 6, 7, 8, 9, 10, 15, 16, 17, 34, 50, 63 mg etc.

The compositions described herein (e.g. defined dose of MT containing compound plus optionally other ingredients) may be provided in a labelled packet along with instructions for their therapeutic or prophylactic use.

In one embodiment, the pack is a bottle, such as are well known in the pharmaceutical art. A typical bottle may be made from pharmacopoeial grade HDPE (High-Density Polyethylene) with a childproof, HDPE push-lock closure and contain silica gel desiccant, which is present in sachets or canisters. The bottle itself may comprise a label, and be packaged in a cardboard container with instructions for use and optionally a further copy of the label.

In one embodiment, the pack or packet is a blister pack (preferably one having aluminium cavity and aluminium foil) which is thus substantially moisture-impervious. In this case the pack may be packaged in a cardboard container with instructions for use and label on the container.

Said label or instructions may provide information regarding the maximum permitted daily dosage of the compositions as described herein—for example based on once daily, b.i.d., or t.i.d.

Said label or instructions may provide information regarding the suggested duration of treatment.

### Salts and Solvates

Although the LMTX containing compounds described herein are themselves salts, they may also be provided in the form of a mixed salt (i.e., the compound of the invention in combination with another salt). Such mixed salts are intended to be encompassed by the term “and pharmaceutically acceptable salts thereof”. Unless otherwise specified, a reference to a particular compound also includes salts thereof.

The compounds of the invention may also be provided in the form of a solvate or hydrate. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, a penta-hydrate etc. Unless otherwise specified, any reference to a compound also includes solvate and any hydrate forms thereof.

Naturally, solvates or hydrates of salts of the compounds are also encompassed by the present invention.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. Treatment effects of LMTM alone or following chronic pretreatment with rivastigmine in wild-type mice on hippocampal levels of acetylcholine (A) or synaptophysin levels measured immunohistochemically as the mean in hippocampus, visual cortex, diagonal band and septum (B). (**, p<0.01; ***, p<0.001).

FIG. 2. Treatment effects of LMTM alone or following chronic pretreatment with rivastigmine in tau transgenic L1 mice on levels of (A) SNARE complex proteins (SNAP25, syntaxin and VAMP2) and (B) α-synuclein measured immunohistochemically as the mean in hippocampus, visual cortex, diagonal band and septum. (*, p<0.05; ***, p<0.001; ****, p<0.0001).

### EXAMPLES

### Example 1—Provision of MT-Containing Compounds

Methods for the chemical synthesis of the MT-containing compounds described herein are known in the art. For example:

Synthesis of compounds 1 to 7 can be performed according to the methods described in WO2012/107706, or methods analogous to those. Synthesis of compound 8 can be performed according to the methods described in WO2007/110627, or a method analogous to those.

### Example 2—Features of the Tau Transgenic Mouse Model Used for Interference Studies

In the L1 mouse model which was used in some of the present studies, there is over-expression of a three-repeat tau fragment encompassing residues 296-390 of the 2N4R tau isoform under the control of the Thy 1 promotor in an NMRI mouse strain (WO2002/059150). This fragment corresponds to the segment of tau first identified within the proteolytically stable core of the PHF (Wischik et al., 1988a; Wischik et al., 1988b) and recently confirmed by cryo-electronmicroscopy of PHFs in AD and tau filaments in Pick's disease (Fitzpatrick et al., 2017; Falcon et al., 2018).

Further features of the L1 mouse model include a prominent loss of neuronal immunoreactivity for choline acetyltransferase in the basal forebrain region, and a corresponding reduction in acetylcholinesterase in neocortex and hippocampus, indicative of reduction in acetylcholine. There is also an approximate 50% reduction in glutamate release for brain synaptosomal preparations from L1 mice compared with those from wild-type mice. In these respects, therefore, L1 mice also model the neurochemical impairments in cholinergic (Mesulam, 2013; Pepeu and Grazia Giovannini, 2017) and glutamatergic (Revett et al., 2013) function that are characteristic of AD and also in other synucleinopathies.

Underlying these impairments in neurotransmitter function, the L1 mouse model shows a disturbance in integration of synaptic proteins. Quantitative immunohistochemistry for multiple synaptic proteins in the basal forebrain (vertical diagonal band) shows that there is normally a high degree of correlation in levels of proteins comprising the SNARE complex (e.g. SNAP-25, syntaxin, VAMP2; reviewed in Li and Kavalali, 2017), and the vesicular glycoprotein synaptophysin and α-synuclein in wild-type mice. These correlations are largely lost in L1 mice (Table 1). The only correlations that remain are between synaptophysin, syntaxin and VAMP2. Therefore, synaptic vesicular protein levels are no longer linked quantitatively to the proteins of the SNARE complex or α-synuclein. This suggests that the tau oligomer pathology of the L1 mice interferes with the functional integration between vesicular and membrane-docking proteins in the synapse.

### Example 3—Experimental Paradigms, Results and Discussion

### Experimental Paradigms

The treatment schedule used to study the negative interaction between symptomatic treatments and LMTM was designed to model the clinical situation in which subjects are first treated chronically with a cholinesterase inhibitor or memantine before receiving LMTM. In what follows, we summarise some of the key results obtained for the AChEI, rivastigmine.

Wild-type and L1 mice (n=7-16 for each group) were pre-treated with rivastigmine (0.1 or 0.5 mg/kg/day) or memantine (2 or 20 mg/kg/day) or vehicle for 5 weeks by gavage. For the following 6 weeks, LMTM (5 and 15 mg/kg) or vehicle were added to this daily treatment regime, also by gavage. Animals were tested behaviourly during weeks 10 and 11 using a problem solving task in the open field water maze and then sacrificed for immunohistochemical and other tissue analyses.

Translating doses from mice to humans requires consideration of a number of factors. Although 5 mg/kg/day in mice corresponds approximately to 8 mg/day in humans in terms of Cₘₐₓ levels of parent MT in plasma, this dose is at the threshold for effects on pathology and behaviour. The higher dose of 15 mg/kg/day is generally required for LMTM to be fully effective in the L1 mouse model (Melis et al., 2015a). This may relate to the much shorter half-life of MT in mice (4 hours) compared to humans (37 hours in elderly humans). Tissue sectioned for immunohistochemistry was labelled with antibody and processed using Image J to determine protein expression densitometrically. Data are presented as Z-score transformations without units.

For measurement of acetylcholine (ACh) levels in hippocampus, animals (wild-type or L1) were treated with LMTM (5 mg/kg/day for 2 weeks) after prior treatment for 2 weeks with or without rivastigmine (0.5 mg/kg/day). Rivastigmine was administered subcutaneously with an Alzet minipump whereas LMTM was administered by oral gavage. Levels of ACh were measured in hippocampus using an implanted microdialysis probe and HPLC analysis of the extracellular fluid.

Data are presented as group averages and standard errors of mean and were analysed using parametric statistics, with alpha set to 0.05.

Experiments on animals were carried out in accordance with the European Communities Council Directive (63/2010/EU) with local ethical approval, a project license under the UK Scientific Procedures Act (1986), and in accordance with the German Law for Animal Protection (Tierschutzgesetz) and the Polish Law on the Protection of Animals.

### Results

Effects of Treatment with LMTM and Rivastigmine in Wild-Type Mice

The effects of treatment with LMTM alone or on a chronic rivastigmine background are summarised in Table 2.

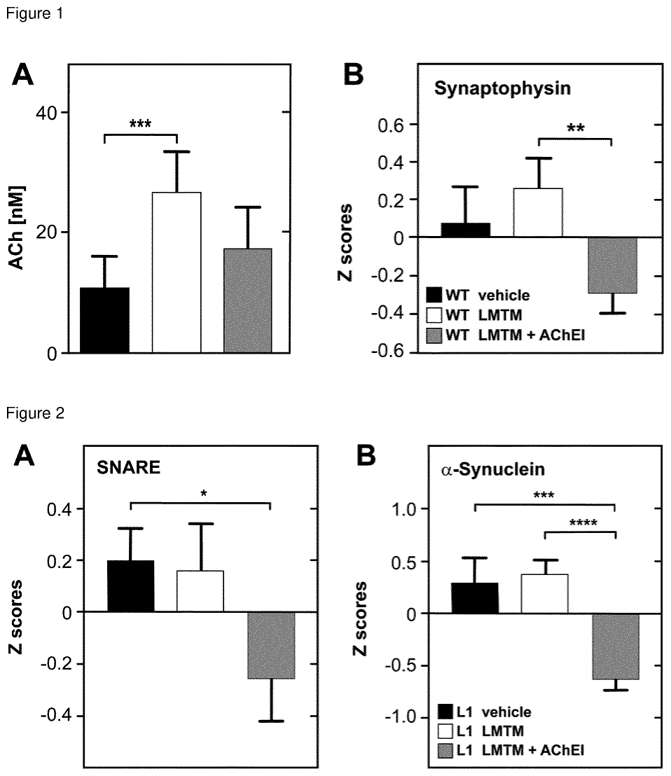

In wild-type mice, there was a significant, 2-fold increase in basal ACh levels in hippocampus following LMTM treatment, and a 30% reduction when mice received LMTM after prior treatment with rivastigmine (FIG. 1A).

There was also a 3-fold increase in mean synaptophysin levels measured in hippocampus, visual cortex, diagonal band and septum following LMTM treatment alone and a statistically significant reduction of the same magnitude when LMTM was given against a background of prior treatment with rivastigmine (FIG. 1B).

TABLE 2
Summary of treatment effects of LMTM given alone (5 or 15 mg/kg/day)
or following chronic pretreatment with rivastigmine (0.1 or 0.5
mg/kg/day) in wild-type mice, given as approximate rounded percentages
to indicate scale and direction of change. Numbers in black signify
treatment effects which reached statistical significance, those
in grey were directional, ‘—’ indicates no effect.

| | | Rivastigmine + |
|---|---|---|
| Effects in wild-type mice | LMTM alone | LMTM |
| ACh release | ↑ × 200% | ↓ × 30% |
| SNARE complex | — | — |
| Synaptophysin | ↑ × 300% | ↓ × 300% |
| α-Synuclein | — | — |
| Mitochondrial complex IV | — | — |
| Behaviour | — | — |

Effects of Treatment with LMTM and Rivastigmine in Tau Transgenic L1 Mice

The activating effects of LMTM alone and the inhibitory effects of the combination with rivastigmine are larger and more generalised in the tau transgenic L1 mice than in the wild-type mice (see Table 3). LMTM alone produces significant increases in ACh release in the hippocampus, in glutamate release from brain synaptosomal preparations, in synaptophysin levels, in mitochondrial complex IV activity and in behavioural changes. None of these effects were seen when LMTM was preceded by chronic rivastigmine. Indeed, in the case of SNARE complex proteins (FIG. 2A) and synuclein (FIG. 2B), the reduction produced by the combination was to levels below those seen in the absence of LMTM treatment.

TABLE 3
Summary of treatment effects of LMTM given alone (5 or 15
mg/kg/day) or following chronic pretreatment with rivastigmine
(0.1 or 0.5 mg/kg/day) in L1 mice, given as approximate rounded
percentages to indicate scale and direction of change. Numbers
in black signify treatment effects that reached statistical
significance, those in grey were directional and n/a signifies
that results are not yet available.

| | | Rivastigmine + |
|---|---|---|
| Effects in L1 mice | LMTM alone | LMTM |
| ACh release | ↑ × 200% | ↓ × 30% |
| Glutamate release | ↑ × 200% | n/a |
| SNARE complex | — | ↓ × 300% |
| Synaptophysin | ↑ × 400% | ↓ × 300% |
| α-Synuclein | — | ↓ × 200% |
| Mitochondrial complex IV | ↑ × 50% | ↓ × 30% |
| Behaviour | ↑ × 30% | ↓ × 20% |

### Discussion of Example 3

The results presented here demonstrate that the reduction in efficacy of LMTM when given as an add-on to a symptomatic treatment in humans can be reproduced both in wild-type mice and in a tau transgenic mouse model.

The results we now report demonstrate that there are two classes of effect produced by LMTM treatment in wild-type and tau transgenic mice: those that are subject to dynamic modulation by prior exposure to cholinesterase inhibitor and those which are not. In tau transgenic mice, the treatment effects that can be modulated include increase in ACh release in the hippocampus, changes in synaptic proteins, increase in mitochondrial complex IV activity and reversal of behavioural impairment. The only treatment effects that are not subject to pharmacological modulation are the primary effect on tau aggregation pathology and its immediate effect on neuronal function, as measured for example by restoration of choline acetyltransferase expression in the basal forebrain.

Effects that are subject to pharmacological modulation are themselves of two types: those which are augmented by the effect on tau aggregation pathology and those which are also seen in wild-type mice. Of the outcomes we have measured, positive treatment effects of LMTM given alone in wild-type mice included an increase in ACh levels in hippocampus, and an increase in synaptophysin levels in multiple brain regions. Therefore, LMTM treatment is able to activate neuronal function at therapeutically relevant doses in wild-type mice lacking tau aggregation pathology.

An increase in synaptophysin signals an increase in number or size of the synaptic vesicles that are required for release of neurotransmitters from the presynapse following activation via an action potential. Therefore, an increase in synaptophysin levels appears to be associated with an increase in a number of neurotransmitters needed to support cognitive and other mental functions.

Although it has been reported that the MT moiety is a weak cholinesterase inhibitor (Pfaffendorf et al., 1997; Deiana et al., 2009), this is unlikely to be the mechanism responsible for the increase in ACh levels.

Specifically, further experiments using scopolamine to increase ACh levels (by blocking M2/M4 negative feedback receptors) showed that the increase produced by LMTM was less than that seen with rivastigmine alone, and that the combination was again inhibitory in wild type mice. Under the condition of cholinesterase inhibition used in these experiments (a very small amount of a cholinesterase inhibitor, 100 nanomolar rivastigmine, added to the perfusion fluid), ACh levels in the hippocampus rise, and when they rise strongly enough, they limit additional ACh release by activating pre-synaptic muscarinic receptors of the M2/M4 subtype (so-called negative feedback receptors).

In this situation, adding scopolamine (1 μM) to the perfusion fluid blocks these presynaptic receptors, and as a consequence, ACh levels rise by 3-5 fold. The fact that LMTM is not additive with rivastigmine in these experiments supports the conclusion that LMTM has a different mechanism of action from rivastigmine. In other words, although LMTM has been described as being a weak inhibitor of cholinesterases in high concentrations, the present effects seem to be unrelated to cholinesterase inhibition, because there is no additive effect with small quantities of rivastigmine.

The increase in ACh and synaptophysin levels might theoretically be explained by an increase in presynaptic mitochondrial activity, since the MT moiety is known to enhance mitochondrial complex IV activity (Atamna et al., 2012), and mitochondria have an important role in homeostatic regulation of presynaptic function (Devine and Kittler, 2018). In particular, The MT moiety is thought to enhance oxidative phosphorylation by acting as an electron shuttle between complex I and complex IV (Atamna et al., 2012). The MT moiety has a redox potential of approximately 0 mV, midway between the redox potential of complex I (−0.4 mV) and complex IV (+0.4 mV).

However, direct measurement of complex IV activity in wild type mice did not show any increase following LMTM treatment. The activating effects of LMTM were also not associated with improvement in spatial recognition memory in wild-type mice.

Although qualitatively similar, the effects of LMTM given alone are much more prominent and more broad-ranging in tau transgenic L1 mice. The most likely explanation for this is that LMTM combines an inhibitory effect on tau oligomers together with inherent activating effects which are not tau-dependent. The reduction in tau oligomer levels following LMTM treatment facilitates a more pronounced activation of synaptic function and release of neurotransmitters such as ACh and glutamate. Likewise, LMTM reverses the spatial memory deficit seen in tau transgenic L1 mice (Melis et al., 2015a). Alternatively, LMTM may act via a different mechanism that does not depend on tau, as seen for example in wild-type mice lacking tau pathology. The negative effects seen when LMTM is introduced on a chronic rivastigmine background appears simply to reflect the reversal of the activation seen with LMTM alone.

A deleterious effect of tau oligomers on functioning of synaptic proteins is readily understandable as being the result of direct interference with docking of synaptic vesicles, membrane fusion and release of neurotransmitter. In tau transgenic L1 mice for example, synaptic vesicular protein levels are no longer linked quantitatively to either the proteins of the SNARE complex or α-synuclein, implying a loss of functional integration between vesicular and membrane-docking proteins at the synapse. The consequence of this can be seen directly as an impairment in glutamate release from synaptosomal preparations from tau transgenic mice, and a restoration of normal glutamate release following treatment with LMTM.

A further consideration is whether the homeostatic downregulation that we have demonstrated would operate in the same way if LMTM treatment were primary and symptomatic treatment were added at a later date. The experiments we have conducted to date were originally designed to mimic the clinical situation in which LMTM is added in patients already receiving symptomatic treatments. If homeostatic downregulation is determined by the treatment that comes first, it is logical that the treatment effects of LMTM would dominate, albeit that the response to add-on symptomatic treatment could be reduced to some extent.

### Example 4—Synaptopathies

As disclosed herein LMTX compounds are capable of increasing mean levels of synaptic proteins in various brain regions at therapeutically relevant doses both in the impaired and wild-type mice. This increase in synaptic proteins may be used to compensate for loss of integration of synaptic proteins in diseases such as synaptopathies i.e. brain disorders that have arisen from synaptic dysfunction, or in which such synaptic dysfunction contributes to the aetiology or symptoms of the disorder. A non-limiting list of such diseases includes the following:

Schizophrenia is a devastating mental disorder with a complex etiology that arises as an interaction between genetic and environmental factors. Schizophrenia is a neurodevelopmental disorder, and synaptic disturbances play a critical role in developing the disease. In 1982, Feinberg proposed that the schizophrenia might arise as a result of abnormal synaptic pruning. Synaptic disturbances cannot be studied and understood as an independent disease hallmark, but only as a part of a complex network of homeostatic events. Development, glial-neural interaction, changes in energy homeostasis, diverse genetic predisposition, neuroimmune processes and environmental influences all can tip the delicate homeostatic balance of the synaptic morphology and connectivity in a uniquely individual fashion, thus contributing to the emergence of the various symptoms of this devastating disorder. Faludi and Mirnics (2011) have broadly sub-stratified schizophrenia into “synaptic” “oligodendroglial”, “metabolic” and “inflammatory” subclasses.

The level of SNAP-25 is significantly depleted in the schizophrenic cerebellum (Mukaetova-Ladinska et al., 2002). Tau and MAP2 and synaptic proteins other than SNAP25, such as synaptophysin and syntaxin, are not affected. This provides evidence that alterations of the cerebellar synaptic network occur in schizophrenia. These changes may influence cerebellar-forebrain connections, especially those with the frontal lobes, and give rise to the cognitive dysmetria that is characteristic of the clinical phenotype in schizophrenia.

Pregulated formation of SNARE complexes and the abnormal expression of SNARE proteins and accessory molecules in a specific region (orbitofrontal cortex) of the human brain are associated with schizophrenia (Katrancha et al., 2015)

Depression. Atrophy of neurons and the loss of glutamatergic synaptic connections caused by stress are key contributors to the symptoms of depression. In addition to the HPA axis, synaptic number and function are altered by other factors (notably neurotrophic factors) that have been implicated in depression (Duman et al., 2016).

Autism spectrum disorders are a complex group of disorders associated with aberrant synaptic transmission and plasticity (Giovedi et al., 2014). Levels of both postsynaptic homer1 and presynaptic synaptophysin were significantly reduced in the adult brain of a shank3b-deficient zebrafish model of ASD (Liu et al., 2018).

Epilepsy: several synaptic proteins are implicated in epilepsy (Giovedi et al., 2014). Electrical kindling increases synaptophysin immunoreactivity in both the hippocampal formation and the piriform cortex in rats (Li et al., 2002).

Startle disease (hyperekplexia) is a rare non-epileptic disorder characterised by an exaggerated persistent startle reaction to unexpected auditory, somatosensory and visual stimuli, generalised muscular rigidity, and nocturnal myoclonus. The major form has a genetic basis: mutations in the al subunit of the glycine receptor gene, GLRA1, or related genes (Bakker et al., 2006). Related syndromes include Tourette's syndrome and anxiety disorders.

Focal hand dystonia, is a syndrome characterized by muscle spasms giving rise to involuntary movements and abnormal postures. Significant alterations in synaptic plasticity have been described in dystonic animal models as well as in patients (Quartarone and Pisani, 2011).

Cerebral ischemia causes synaptic alterations that are consistent with ischemic long-term potentiation (LTP) and represent a new model to characterize aberrant forms of synaptic plasticity. (Orfila et al., 2018). Although immunoreactivity for synaptophysin is transiently increased in ischemic lesions from 3 to 7 days after cerebral ischemia, synaptophysin immunostaining in the damaged areas gradually decreased and finally almost disappeared one month after transient cerebral ischemia in rats (Korematsu et al., 1993).

The inflammatory cytokines tumor necrosis factor (TNF) and interleukin-1β (IL-1β) play important physiological roles in LTP and synaptic scaling. However, actions of these cytokines on synaptic plasticity can be altered under conditions of neuroinflammation. Altered synaptic plasticity occurs under either physiological or inflammatory conditions, in particular for experimental allergic encephalitis (EAE) and multiple sclerosis (MS) (Rizzo et al. 2018). Synaptophysin, synapsin I, and PSD-95 immunoreactivities were reduced in both the grey and white matter of both chronic and acute models of EAE (Zhu et al., 2013).

Glaucoma and AD share several features. They both affect the elderly, are neurodegenerative, chronic and progressive, leading to irreversible cell death. AD and glaucoma also share some common features such as the Aβ accumulation/aggregation, tau aggregation and hyperphosphorylation. Both diseases are characterized by early changes of neuronal circuitry and phosphorylation of mitogen-activated protein kinases (MAPK) followed by inflammatory process, glial reaction, reactive oxygen species production, oxidative stress and mitochondrial abnormalities, propagation of neurodegenerative processes leading to cell death. Both diseases are characterized by common features such as synaptic dysfunction and neuronal cell death at the level of the inner retina. Glaucoma is recognized as a disease frequently associated with AD and aging (Criscuolo et al., 2017).

### REFERENCES FOR EXAMPLE 4

Bakker, M J, van Dijk, J G, van den Maagdenberg, A M J M, Tijssen, M A J (2006) Startle syndromes. Lancet Neurol. 5:513-524
Criscuolo, C, Fabiani, C, Cerri, E, Domenici, L (2017) Synaptic dysfunction in Alzheimer's disease and glaucoma: from common degenerative mechanisms toward neuroprotection. Frontiers in Cellular Neuroscience 11:53
Duman, R S, Aghajanian, G K, Sanacora, G, Krystal, J H (2016) Synaptic plasticity and depression: new insights from stress and rapid-acting antidepressants. Nature Med. 22:238
Faludi, G, Mirnics, K (2011) Synaptic changes in the brain of subjects with schizophrenia. Int. J. Devel. Neurosci. 29:305-309
Giovedi, S, Corradi, A, Fassio, A, Benfenati, F (2014) Involvement of synaptic genes in the pathogenesis of autism spectrum disorders: the case of synapsins. Frontiers in Pediatrics 2:94
Korematsu, K, Goto, S, Nagahiro, S, Ushio, Y (1993) Changes of immunoreactivity for synaptophysin (‘protein p38’) following a transient cerebral ischemia in the rat striatum. Brain Res. 616:320-324
Lepeta, K, Lourenco, M V, Schweitzer, B C, Martino Adami, P V, Banerjee, P et al. (2016) Synaptopathies: synaptic dysfunction in neurological disorders. J. Neurochem. 139:785-805
Liu, C-x, Li, C-y, Hu, C-c, Wang, Y, Lin, J et al. (2018) CRISPR/Cas9-induced shank3b mutant zebrafish display autism-like behaviors. Molecular Autism 9:23
Mukaetova-Ladinska, E B, Hurt, J, Honer, W G, Harrington, C R, Wischik, C M (2002) Loss of synaptic but not cytoskeletal proteins in the cerebellum of chronic schizophrenics. Neurosci. Lett. 317:161-165
Orfila, J E, McKinnon, N, Moreno, M, Deng, G, Chalmers, N et al. (2018) Cardiac arrest induces ischemic long-term potentiation of hippocampal CA1 neurons that occludes physiological long-term potentiatvion. Neural Plasticity 2018:9275239
Poirel, O, Mella, S, Videau, C, Ramet, L, Davoli, M A et al. (2018) Moderate decline in select synaptic markers in the prefrontal cortex (BA9) of patients with Alzheimer's disease at various cognitive stages. Sci. Rep. 8:938
Quartarone, A, Pisani, A (2011) Abnormal plasticity in dystonia: Disruption of synaptic homeostasis. NeurobioL Dis. 42:162-170
Rizzo, F R, Musella, A, De Vito, F, Fresegna, D, Bullitta, S et al. (2018) Tumor necrosis factor and interleukin-1□ modulate synaptic plasticity during neuroinflammation. Neural Plasticity 2018:8430123
Sze, C-I, Troncoso, J C, Kawas, C, Mouton, P, Price, D L, Martin, L J (1997) Loss of the presynaptic vesicle protein synaptophysin in hippocampus correlates with cognitive decline in Alzheimer disease. J. Neuropathol. Exptl. Neurol. 56:933-944
Terry, R D, Masliah, E, Salmon, D P, Butters, N, DeTeresa, R et al. (1991) Physical basis of cognitive alterations in Alzheimer's disease: synapse loss is the major correlate of cognitive impairment. Ann. Neurol. 30:572-580
Giau, V V, Senanarong, V, Bagyinszky, E, An, S S A, Kim, S (2019) Analysis of 50 neurodegenerative genes in clinically diagnosed early-onset Alzheimer's disease. International Journal of Molecular Sciences 20:1514
Zhu, B, Luo, L, Moore, G R W, Paty, D W, Cynader, M S (2003) Dendritic and synaptic pathology in experimental autoimmune encephalomyelitis. Am. J. Pathol. 162:1639-1650

### OTHER REFERENCES

Al-Hilaly, Y. K., Pollack, S. J., Rickard, J. E., Simpson, M., Raulin, A.-C., Baddeley, T., et al. (2018). Cysteine-independent inhibition of Alzheimer's disease-like paired helical filament assembly by leuco-methylthioninium (LMT). J. Mol. Biol. 430, 4119-4131. doi: 10.1016/j.jmb.2018.08.010.
Atamna, H., Mackey, J., and Dhahbi, J. M. (2012). Mitochondrial pharmacology: electron transport chain bypass as strategies to treat mitochondrial dysfunction. Biofactors 38, 158-166. doi: 10.1002/biof.197.
Baddeley, T. C., Mccaffrey, J., Storey, J. M. D., Cheung, J. K. S., Melis, V., Horsley, D., et al. (2015). Complex disposition of methylthioninium redox forms determines efficacy in tau aggregation inhibitor therapy for Alzheimer's disease. J. Pharmacol. Exptl. Therapeutics 352, 110-118. doi: 10.1124/jpet.114.219352.
Callaway, N. L., Riha, P. D., Bruchey, A. K., Munshi, Z., and Gonzalez-Lima, F. (2004). Methylene blue improves brain oxidative metabolism and memory retention in rats. Pharmacol. Biochem. Behay. 77, 175-181.
Callaway, N. L., Riha, P. D., Wrubel, K. M., Mccollum, D., and Gonzalez-Lima, F. (2002). Methylene blue restores spatial memory retention impaired by an inhibitor of cytochrome oxidase in rats. Neurosci. Lett. 332, 83-86.
Deiana, S., Harrington, C. R., Wischik, C. M., and Riedel, G. (2009). Methylthioninium chloride reverses cognitive deficits induced by scopolamine: comparison with rivastigmine. Psychopharmacology 202, 53-65. doi: 10.1007/s00213-008-1394-2.
Devine, M. J., and Kittler, J. T. (2018). Mitochondria at the neuronal presynapse in health and disease. Nat. Rev. Neurosci. 19, 63-80. doi: 10.1038/nrn.2017.170.
Fitzpatrick, A. W. P., Falcon, B., He, S., Murzin, A. G., Murshudov, G., Garringer, H. J., et al. (2017). Cryo-EM structures of tau filaments from Alzheimer's disease. Nature 547, 185-190. doi: 10.1038/nature23002.
Gauthier, S., Feldman, H. H., Schneider, L. S., Wilcock, G. K., Frisoni, G. B., Hardlund, J. H., et al. (2016). Efficacy and safety of tau-aggregation inhibitor therapy in patients with mild or moderate Alzheimer's disease: a randomised, controlled, double-blind, parallel-arm, phase 3 trial. Lancet 388, 2873-2884. doi: 10.1016/S0140-6736(16)31275-2.
Gonzalez-Lima, F., and Bruchey, A. K. (2004). Extinction memory by the metabolic enhancer improvement methylene blue. Learning & Memory 11, 633-640.
Harrington, C. R., Storey, J. M. D., Clunas, S., Harrington, K. A., Horsley, D., Ishaq, A., et al. (2015). Cellular models of aggregation-dependent template-directed proteolysis to characterize tau aggregation inhibitors for treatment of Alzheimer's disease. J. Biol. Chem. 290, 10862-10875. doi: 10.1074/jbc.M114.616029.
Husain, M., and Mehta, M. A. (2011) Cognitive enhancement by drugs in health and disease. Trends Cognitive Sci. 15, 28-36.
Katrancha, S M, and Koleske, A J (2015) SNARE Complex Dysfunction: A Unifying Hypothesis for Schizophrenia. Biol. Psychiatry 78:356-358.
Li, Y. and Kavalali, E. T. (2017). Synaptic vesicle-recycling machinery components as potential therapeutic targets. Pharmacol. Rev. 69, 141-160.
Maier, L. J., Ferris, J. A., and Winstock, A. R. (2018). Pharmacological cognitive enhancement among non-ADHD individuals—A cross-sectional study in 15 countries.” Int. J. Drug Policy 58, 104-112.
Martinez, J. L., Jr., Jensen, R. A., Vasquez, B. J., Mcguiness, T., and Mcgaugh, J. L. (1978). Methylene blue alters retention of inhibitory avoidance responses. Physiological Psychology 6, 387-390.
Melis, V., Magbagbeolu, M., Rickard, J. E., Horsley, D., Davidson, K., Harrington, K. A., et al. (2015a). Effects of oxidized and reduced forms of methylthioninium in two transgenic mouse tauopathy models. Behav. Pharmacol. 26, 353-368. doi: 10.1097/fbp.0000000000000133.
Melis, V., Zabke, C., Stamer, K., Magbagbeolu, M., Schwab, K., Marschall, P., et al. (2015b). Different pathways of molecular pathophysiology underlie cognitive and motor tauopathy phenotypes in transgenic models for Alzheimer's disease and frontotemporal lobar degeneration. Cell. Mol. Life Sci. 72, 2199-2222. doi: 10.1007/s00018-014-1804-z.
Mesulam, M. M. (2013). Cholinergic circuitry of the human nucleus basalis and its fate in Alzheimer's disease. J. Comp. Neurol. 521, 4124-4144. doi: 10.1002/cne.23415.
Pepeu, G., and Grazia Giovannini, M. (2017). The fate of the brain cholinergic neurons in neurodegenerative diseases. Brain Res. 1670, 173-184. doi: 10.1016/j.brainres.2017.06.023.
Pfaffendorf, M., Bruning, T. A., Batink, H. D., and Van Zwieten, P. A. (1997). The interaction between methylene blue and the cholinergic system. Br. J. Pharmacol. 122, 95-98. doi: 10.1038/sj.bjp.0701355.
Revett, T. J., Baker, G. B., Jhamandas, J., and Kar, S. (2013). Glutamate system, amyloid beta peptides and tau protein: functional interrelationships and relevance to Alzheimer disease pathology. J. Psychiat. Neurosci. 38, 6-23. doi: 10.1503/jpn.110190.
Riha, P. D., Bruchey, A. K., Echevarria, D. J., and Gonzalez-Lima, F. (2005). Memory facilitation by methylene blue: Dose-dependent effect on behavior and brain oxygen consumption. Eur. J. Pharmacol. 511, 151-158.
Wilcock, G. K., Gauthier, S., Frisoni, G. B., Jia, J., Hardlund, J. H., Moebius, H. J., et al. (2018). Potential of low dose leuco-methylthioninium bis(hydromethanesulphonate) (LMTM) monotherapy for treatment of mild Alzheimer's disease: cohort analysis as modified primary outcome in a phase 3 clinical trial. J. Alzheimer's Dis. 61, 635-657. doi: 10.3233/JAD-170560.
Wischik, C. M., Edwards, P. C., Lai, R. Y. K., Roth, M., and Harrington, C. R. (1996). Selective inhibition of Alzheimer disease-like tau aggregation by phenothiazines. Proc. Natl. Acad. Sci. U.S.A. 93, 11213-11218. doi: 10.1073/pnas.93.20.11213.
Wischik, C. M., Novak, M., Edwards, P. C., Klug, A., Tichelaar, W., and Crowther, R. A. (1988a). Structural characterization of the core of the paired helical filament of Alzheimer disease. Proc. Natl. Acad. Sci. U.S.A. 85, 4884-4888. doi: 10.1073/pnas.85.13.4884.
Wischik, C. M., Novak, M., Thogersen, H. C., Edwards, P. C., Runswick, M. J., Jakes, R., et al. (1988b). Isolation of a fragment of tau derived from the core of the paired helical filament of Alzheimer disease. Proc. Natl. Acad. Sci. U.S.A. 85, 4506-4510. doi: 10.1073/pnas.85.12.4506.
Wischik, C. M., Schelter, B. O., Wischik, D. J., Storey, J. M. D., and Harrington, C. R. (2018). Modeling prion-like processing of tau protein in Alzheimer's disease for pharmaceutical development. J. Alzheimer's Dis. 62, 1287-1303. doi: 10.3233/JAD-170727.
Wrubel, K. M., Barrett, D., Shumake, J., Johnson, S. E., and Gonzalez-Lima, F. (2007). Methylene blue facilitates the extinction of fear in an animal model of susceptibility to learned helplessness. Neurobiol. Learning Memory 87, 209-217.
Zoellner, L. A., Telch, M., Foa, E. B., Farach, F. J., Mclean, C. P., Gallop, R., et al. (2017). Enhancing extinction learning in posttraumatic stress disorder with brief daily imaginal exposure and methylene blue: a randomized controlled trial. J. Clin. Psychiat. 78, e782-e789. doi: 10.4088/JCP.16m10936.

1. A method of increasing the level of synaptophysin in the brain of a mammalian subject,
which method comprises orally administering to said subject a methylthioninium (MT)-containing compound,
wherein the MT-containing compound is an LMTX compound of the following formula: 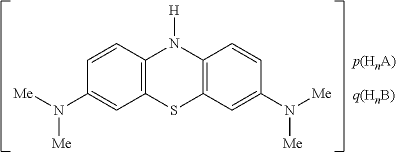
wherein each of HₙA and HₙB (where present) are protic acids which may be the same or different,
and wherein p=1 or 2; q=0 or 1; n=1 or 2; (p+q)×n=2,
or a hydrate or solvate thereof.

2. A method of therapeutic treatment a synaptopathy disorder in a subject which disorder is selected from the list consisting of: schizophrenia; cerebral ischemia; Multiple sclerosis (MS); depression; epilepsy; Startle syndrome; Tourette's syndrome; Autism spectrum disorders (ASD); Focal hand dystonia; Experimental allergic encephalitis (EAE); Glaucoma; late onset Alzheimer's disease synaptic dysfunction type; a Lysozomal storage disease not associated with tau pathology
which method comprises orally administering to said subject a methylthioninium (MT)-containing compound,
wherein the MT-containing compound is an LMTX compound of the following formula: 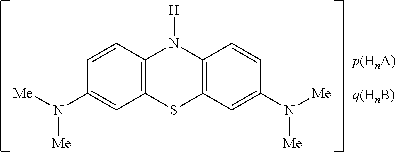
wherein each of HₙA and HₙB (where present) are protic acids which may be the same or different,
and wherein p=1 or 2; q=0 or 1; n=1 or 2; (p+q)×n=2,
or a hydrate or solvate thereof.

3. A method as claimed in claim 1 wherein the treatment is combined with a further therapeutic agent for that disorder.

4. A method as claimed in claim 1 or claim 2 wherein the total daily dose is between 2 and 100 mg of MT, optionally 10-60 mg, to the subject per day, optionally split into 2 or more doses.

5. A method as claimed in claim 4 wherein the total daily dose is from around any of 10, 11, 12, 13, 14, 15, 16, 17, 18, 19, 20 mg to around any of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 mg.

6. A method as claimed in claim 4 wherein the total daily dose is between 20 and 40 mg.

7. A method as claimed in claim 4 wherein the total daily dose is about 20, 21, 22, 23, 24, 25, 26, 27, 28, 29, 30, 31, 32, 33, 34, 35, 36, 37, 38, 39, 40 mg.

8. A method as claimed in any one of claims 1 to 7 wherein the total daily dose of the LMTX compound is administered as a split dose twice a day or three times a day.

9. A method as claimed in any one of claims 1 to 8 wherein the subject has not historically received treatment with a neurotransmission modifying compound which is a modifier of the activity of acetylcholine or glutamate neurotransmitters.

10. A method as claimed in any one of claims 1 to 8 wherein the subject has historically received treatment with the neurotransmission modifying compound which is a modifier of the activity of acetylcholine or glutamate neurotransmitters, but ceased that treatment at least 1, 2, 3, 4, 5, 6, 7 days, or 2, 3, 4, 5, 6, 7, 8 weeks prior to treatment with the LMTX compound.

11. A method as claimed in any one of claims 1 to 8 wherein the subject is selected as one who is receiving treatment with the neurotransmission modifying compound which is a modifier of the activity of acetylcholine or glutamate neurotransmitters, wherein said treatment is discontinued prior to treatment with the LMTX compound.

12. A method as claimed in any one of claims 1 to 11 wherein the therapeutic treatment is not combined with a neurotransmission modifying compound which is a modifier of the activity of acetylcholine or glutamate neurotransmitters.

13. A method as claimed in any one of claims 9 to 12, wherein the neurotransmission modifying compound is an acetylcholinesterase inhibitor.

14. A method as claimed in any one of claims 9 to 13, wherein the neurotransmission modifying compound is selected from donepezil; rivastigmine; and galantamine.

15. A method as claimed in any one of claims 9 to 12, wherein the neurotransmission modifying compound is an N-methyl-D-aspartate receptor (NMDA) receptor antagonist.

16. A method as claimed in any one of claims 9 to 12 or claim 15, wherein the neurotransmission modifying compound is memantine.

17. A method as claimed in any one of claims 1 to 16 wherein the subject is a human who has been diagnosed as having said synaptopathy disorder, or wherein said method comprises making said diagnosis.

18. A method of prophylactic treatment of a synaptopathy disorder in a subject, which disorder is selected from the list consisting of: schizophrenia; cerebral ischemia; Multiple sclerosis (MS); depression; epilepsy; Startle syndrome; Tourette's syndrome; Autism spectrum disorders (ASD); Focal hand dystonia; Experimental allergic encephalitis (EAE); Glaucoma; late onset Alzheimer's disease synaptic dysfunction type; a Lysozomal storage disease not associated with tau pathology,
which method comprises orally administering to said subject a methylthioninium (MT)-containing compound,
wherein the MT-containing compound is an LMTX compound of the following formula: 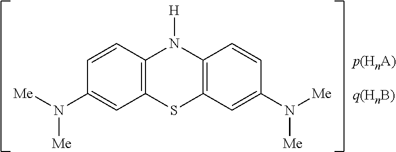
wherein each of HₙA and HₙB (where present) are protic acids which may be the same or different,
and wherein p=1 or 2; q=0 or 1; n=1 or 2; (p+q)×n=2,
or a hydrate or solvate thereof.

19. A method as claimed in claim 18 wherein the treatment is combined with a further prophylactic agent for that disorder.

20. A method as claimed in claim 18 wherein the dose is as defined in any one of claims 4 to 8 and/or the subject is as defined in any one of claims 9 to 11.

21. A method as claimed in any one of claims 18 to 20 wherein the subject is a human who has been assessed as being susceptible to, or at risk of, the disorder, optionally based on familial or genetic or other data.

22. A method as claimed in any one of claims 1 to 21 wherein the LTMX compound has the following formula, where HA and HB are different mono-protic acids:

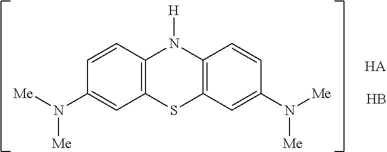

23. A method as claimed in claim 22 wherein the LTMX compound has the following formula: 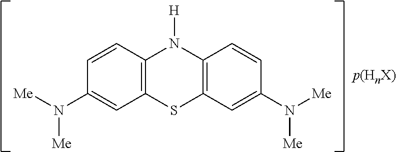
wherein each of HₙX is a protic acid.

24. A method as claimed in claim 22 wherein the LTMX compound has the following formula and H₂A is a di-protic acid:

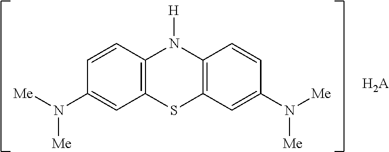

25. A method as claimed in claim 23 wherein the LTMX compound has the following formula and is a bis-monoprotic acid:

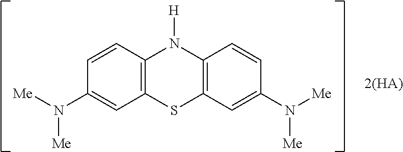

26. A method as claimed in any one of claims 22 to 25 wherein the or each protic acid is an inorganic acid.

27. A method as claimed in claim 26 wherein each protic acid is a hydrohalide acid.

28. A method as claimed in claim 26 wherein the or each protic acid is selected from HCl; HBr; HNO₃; H₂SO₄.

29. A method as claimed in any one of claims 22 to 25 wherein the or each protic acid is an organic acid.

30. A method as claimed in claim 29 wherein the or each protic acid is selected from H₂CO₃; CH₃COOH; methanesulfonic acid, 1,2-ethanedisulfonic acid, ethansulfonic acid, naphthalenedisulfonic acid, p-toluenesulfonic acid.

31. A method as claimed in any one of claims 1 to 30 wherein the LTMX compound is LMTM:

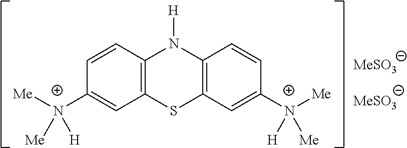

32. A method as claimed in claim 31 wherein the total daily dose of LMTM is around 34 to 67, 34 to 100, 34 to 134, or 34 to 167 mg/day.

33. A method as claimed in claim 32 wherein the dose of LMTM is about 34, 38, 67, or 100 mg/once per day.

34. A method as claimed in any one of claims 1 to 21 wherein the LTMX compound is selected from the list consisting of:

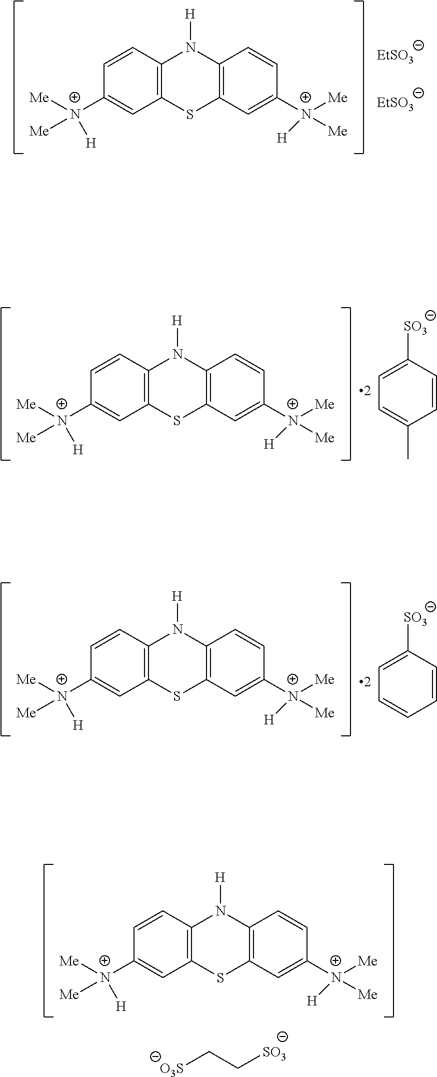

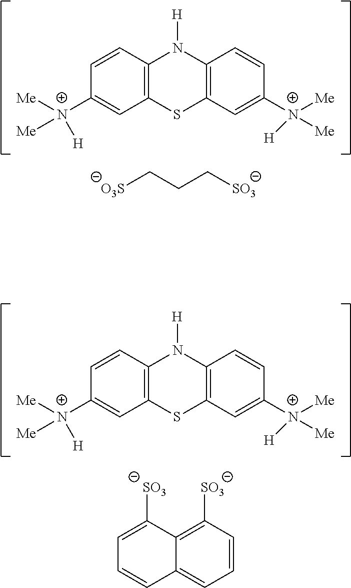

35. A method as claimed in any one of claims 1 to 34 wherein the LTMX compound is provided as a pharmaceutical composition comprising the LMTX compound and a pharmaceutically acceptable carrier or diluent in the form of a dosage unit.

36. A method as claimed in claim 35 wherein the amount of MT in the unit is about 4, 5, 6, 7, 8, 9, 10, 20, or 30 to about 40, 50 or 60 mg.

37. A method as claimed in claim 35 wherein the dosage unit comprises about 34 to 67 mg, 34 to 100, 34 to 134, or 34 to 167 LMTM.

38. A method as claimed in any one of claims 35 to 37 wherein the composition is a tablet or capsule.

39. A container comprising:
(i) a plurality of dosage units as defined in any one of claims 35 to 38;
(ii) a label and/or instructions for their use according to a method of treatment as defined in any one of claims 1 to 34.

40. A container as claimed in claim 39, wherein the container comprises dosage units, and the dosage units are present in a blister pack which is substantially moisture-impervious.

41. A container as claimed in claim 39 or claim 40 wherein the label or instructions provide information regarding the disorder for which the composition is intended.

42. A container as claimed in any one of claims 39 to 41 wherein the label or instructions provide information regarding the maximum permitted daily dosage of the dosage units.

43. A container as claimed in any one of claims 39 to 42 wherein the label or instructions provide information regarding the suggested duration of the treatment.

44. An LTMX compound or composition as defined in any one of claims 1 to 34, for use in a method of treatment as defined in any one of claims 1 to 38.

45. Use of an LTMX compound or composition as defined in any one of claims 1 to 34, in the manufacture of a medicament for use in a method of treatment as defined in any one of claims 1 to 38.
